(19) United States
(12) Patent Application Publication
(10) Pub. No.: US 2019/0142407 A1
(43) Pub. Date: May 16, 2019

(54) METHOD OF UNILATERAL BIPORTAL ENDOSCOPY AND DIAMOND SHAVER USED IN SAME

(71) Applicants: Min Ho Jung, Daegu (KR); Sang-Kyu Son, Busan (KR); ENDOVISION CO., LTD., Daegu (KR)

(72) Inventors: Min Ho Jung, Daegu (KR); Sang-Kyu Son, Busan (KR)

(73) Assignees: ENDOVISION CO., LTD., Daegu (KR); Min Ho Jung, Daegu (KR); Sang-Kyu Son, Busan (KR)

(21) Appl. No.: 15/879,808

(22) Filed: Jan. 25, 2018

(30) Foreign Application Priority Data
Nov. 14, 2017 (KR) 10-2017-0151636
Jan. 22, 2018 (KR) 10-2018-0007729

(51) Int. Cl. A61B 17/02; A61F 2/46; A61B 18/14; A61B 1/045; A61B 1/06; A61B 17/16; A61B 17/00

(52) U.S. Cl. CPC A61B 17/025; A61F 2002/4635; A61F 2/4644; A61B 18/14; A61B 1/045; A61B 1/0661; A61B 17/1659; A61B 17/1615; A61B 17/00234; A61B 2017/00429; A61B 2017/00876; A61B 2217/007; A61B 2017/00862; A61B 2017/00477; A61B 2017/00017; A61B 2217/005; A61B 2017/0256; A61F 2/4611

(57) ABSTRACT

The present invention relates generally to a method of unilateral biportal endoscopy and diamond shaver used in the same. The method includes: securing pathways for a working portal and an endoscopic portal that extend toward a surgical site in the body of a patient and are distanced from each other; inserting a surgical instrument required for surgery into the secured working portal; inserting an endoscope into the endoscopic portal; performing surgery using the surgical instrument inserted into the working portal while monitoring the surgical site through the endoscope; removing the surgical instrument and the endoscope after the performing the surgery; and suturing of suturing entrances of the working portal and the endoscopic portal.

### CROSS REFERENCE TO RELATED APPLICATION

The present application claims priority to Korean Patent Application Nos. 10-2017-0151636 and 10-2018-0007729, filed on Nov. 14, 2017 and Jan. 22, 2018 respectively, the entire contents of which is incorporated herein for all purposes by this reference.

### BACKGROUND OF THE INVENTION

### Field of the Invention

The present invention relates generally to a method of unilateral biportal endoscopy and a diamond shaver used in the same. More particularly, the present invention relates to a method of unilateral biportal endoscopy which separately secures a working portal for surgical instruments and an endoscopic portal for an endoscope, thereby providing a more accurate spinal surgery, and to a diamond shaver which can be effectively applied to the method.

### Description of the Related Art

The intervertebral disc lies between adjacent vertebrae and functions to absorb and distribute the loads of the body and impact, as well as functioning to hold the vertebrae together, and functioning to separate the vertebrae from each other such that the size of the intervertebral foramen is maintained and thus the spinal nerve is not compressed.

However, aging, excessive exercise, habitual poor posture, etc. causes narrowing of the intervertebral foramen. More precisely, the intervertebral foramen becomes narrower due to joint hypertrophy in the spinal motion segment, intervertebral disc degeneration, proinflammatory cytokine released from injured intervertebral disc or cartilage, fibrous adhesion to nerve branches in the intervertebral foramen and to surrounding ligaments, etc.

When the intervertebral foramen becomes narrower, the nerve roots exiting the intervertebral foramen from the spinal cord are compressed. Such compression may cause disc disease symptoms to appear throughout the body, such as pain in the neck, shoulders, back, arms, etc., weakness of muscles, impaired walking, bowel and bladder dysfunction, etc. In particular, foraminal stenosis due to aging mainly results from the narrowing of the intervertebral foramen, which is caused by a bulging disc and posterior longitudinal ligament and hypertrophy of the facet joint and the ligamentum flavum.

As a surgical procedure for performing the above-described spinal disease, a conventional incision is a method of making a large incision in a surgical site. Thus, the conventional incision has a high probability of damaging the blood vessels as well as the spinal nerves and muscles, causes a large amount of bleeding, and has a long recovery period.

In order to solve such a problem, recently, percutaneous stenoscopic lumbar decompression (PSLD), which is a minimally invasive spinal surgical method, has been performed. However, the PSLD itself is a challenging procedure, and an operator may suffer from technical difficulties due to a restricted field of vision despite using a microscope or spinal endoscope as a supplementary device.

On the other hand, nerve branches entrapped by fibrous adhesion can be treated to some degree by only epidural block or epidural neurolysis in the stage of weak adhesions or mild stenosis.

However, when the adhesion or stenosis is severe, approach to the intervertebral foramen is difficult with the procedure described above, or even when treatment is performed after approaching to the intervertebral foramen, there is a high possibility that the pain will recur as a treated area becomes clogged again.

There is percutaneous foraminotomy as the most effective treatment method that can be applied in such circumstances. Percutaneous foraminotomy is a surgical procedure whereby an enlarging device is directly inserted into the intervertebral foramen through the patient's skin, and adhesive fibrosis or bone spurs compressing nerve branches exiting the intervertebral foramen are removed and thus the pain is resolved, thereby relieving the compression applied to the blood vessels in the intervertebral foramen and improving the blood flow around nerves.

For such percutaneous foramnotomy, Korean Patent No. 10-1302453 entitled “percutaneous extraforaminotomy with foraminal ligament resection and instrument tools being used for the same” is disclosed.

A surgical instrument introduced in the document of the related art is used for expanding the intervertebral foramen by removing fibrous adhesion, etc. which block the intervertebral foramen, and is configured such that a trocar inserted into a target point through the skin, a cannula guided by the trocar and securing a pathway, an end mill passing through a guide hole of the cannula and having at an end thereof a blade tip, and a curette having a scraping tip inserted into the guide hole and scraping tissue inside the intervertebral foramen.

However, the above-described conventional surgical instrument is problematic in that a structure thereof is simple and thus operative effects other than detaching tissue at a target point and scrapping the detached tissue may not occur, which may not suitable for an accurate procedure. For example, the blade tip may severely damage normal tissue or touch the blood vessels, leading to internal bleeding.

In addition, the use of the end mill itself is very inconvenient. In order to use the end mill, that is, in order to rotate the blade tip, an operator must hold a handle of the end mill and rotate it to the left and right by applying a force as if rotating a gimlet to the left and right, resulting in operator inconvenience. Moreover, a rotational speed of the blade tip is low, so that adherent tissue may not be cut but torn. Of course, normal tissue also may be torn while the adherent tissue is torn.

Further, grinding a hard mass such as bone spurs may be impossible. In order to cut the hard mass, the blade tip of the end mill must be strongly pressed against a target surface, so that the blade tip may accidentally slip to the other side, causing a serious problem.

### SUMMARY OF THE INVENTION

Accordingly, the present invention has been made keeping in mind the above problems occurring in the related art, and the present invention provides a method of unilateral biportal endoscopy, which is capable of securing a clear field of vision, thereby enabling accurate identification and removal of lesion and securing high safety. In addition, the method enables minimum invasion with fewer scars and less risk of muscle damage, bleeding, and infection, thereby achieving a rapid therapeutic effect.

Further, the present invention provides a diamond shaver used in unilateral biportal endoscopy, the diamond shaver being configured to shave and remove a target and having a drill with a burr embedded with diamond powder, whereby the diamond shaver is excellent in fine cutting ability and thus enables high surgical precision. Thus, a risk of bleeding due to overcutting is reduced, thereby reducing the surgical time.

In order to achieve the above object, according to one aspect of the present invention, there is provided a method of unilateral biportal endoscopy, the method including: securing pathways for a working portal and an endoscopic portal that extend toward a surgical site in the body of a patient and are distanced from each other; inserting a surgical instrument required for surgery into the secured working portal; inserting an endoscope into the endoscopic portal; performing surgery using the surgical instrument inserted into the working portal while monitoring the surgical site through the endoscope; removing the surgical instrument and the endoscope after performing the surgery; and suturing entrances of the working portal and the endoscopic portal.

The securing the pathways may include: marking positions of the entrances of the working portal and the endoscopic portal on the skin of the patient; incising marking portions marked by the marking; inserting an enlarging tube into the body through an incision opened by the incising, thereby forming a pathway extending toward the surgical site; and enlarging the pathway to enlarge a diameter of the pathway by using enlarging tubes having various sizes.

The method may further include: supplying the saline solution supplied from outside to the surgical site and discharging materials at the surgical site from the body, during the performing the surgery.

The working portal and the endoscopic portal may be configured such that the entrances thereof are distanced from each other, and the portals may extend into the body to be close to each other such that ends thereof meet with each other at the surgical site.

An angle between the working portal and the endoscopic portal may be equal to or less than 90 degrees.

According to another aspect of the present invention, there is provided a diamond shaver used in unilateral biportal endoscopy, the diamond shaver being configured to be inserted into a working portal and to shave and remove a target located at the surgical site, the working portal extending toward a surgical site of a patient together with an endoscopic portal, the diamond shaver including: a drill including an outer tube having an inclined opening inclined at a front end thereof to form an acute angle with respect to a lengthwise direction of the outer tube, a tube holder fixed to a rear end of the outer tube, a handpiece adapter provided at a rear side of the tube holder, and a drill body rotatably provided in the outer tube, the drill body including a rotatable shaft portion extending in a lengthwise direction thereof, and a head portion fixed to a front end of the rotatable shaft portion and having a burr embedded with diamond powder; a handpiece detachably coupled to the handpiece adapter and manipulated by an operator; a controller connected to the handpiece and outputting a control signal; and a foot switch connected to the controller and manipulated by an operator's foot.

The burr may be configured to be partially exposed to outside of the outer tube through the inclined opening, such that the burr is brought into contact with a target to be removed.

The electric shaft portion may be provided with: a hollow shaft extending in a lengthwise direction thereof; and a tip portion provided at a front end of the hollow shaft and provided with a coupling hole such that the tip portion is coupled with the head portion, and the head portion may be provided with: a connection portion detachably coupled with the coupling hole; and a burr supporting portion integrally provided with the connection portion and supporting the burr.

The burr supporting portion may be provided with: a tapered body receiving a rotational force from the adapter; and an elastic supporting member being elastically deformable and connecting the tapered body and the burr to each other.

The elastic supporting member may include: a cylindrical torsion rubber or a coil spring having a predetermined diameter.

A fluid guiding portion may be provided between the tube holder and the handpiece adapter, and may allow a saline solution to pass therethrough.

In the present invention, the method of unilateral biportal endoscopy is capable of securing a clear field of vision, thereby enabling accurate identification and removal of a lesion and securing high safety. In addition, the method enables minimal incision with fewer scars and less risk of muscle damage, bleeding, and infection, thereby achieving a rapid therapeutic effect.

Moreover, in the present invention, the diamond shaver is configured to shave and remove a target during unilateral biportal endoscopic surgery and has the burr embedded with diamond powder, whereby the diamond shaver is excellent in fine cutting ability and thus enables high surgical precision. Thus, a risk of bleeding due to overcutting is reduced, thereby reducing the surgical time.

### BRIEF DESCRIPTION OF THE DRAWINGS

FIG. 1 is a view showing a basic concept of a method of unilateral biportal endoscopy;

FIGS. 2A and 2B are views showing a tool kit shown in FIG. 1;

FIGS. 3A to 3D are perspective views showing a root retractor shown in FIG. 2A;

FIG. 4 is a view showing a cage guider shown in FIG. 2A;

FIG. 5 is a perspective view showing a bone chip cannula shown in FIG. 2A;

FIG. 6 is a view showing an osteotome shown in FIG. 2A;

FIG. 7 is a perspective view showing a bone chip impactor shown in FIG. 2A;

FIGS. 8A to 8C are views showing an end plate remover shown in FIG. 2A;

FIG. 9 is a partial perspective view showing a radiofrequency probe shown in FIG. 1;

FIG. 10 is a perspective view showing a K-punch shown in FIG. 1;

FIG. 11 is a view showing an overall configuration of a drill and a bone tissue removing device having the same according to an embodiment of the present invention;

FIG. 12 is a cutaway perspective view showing an internal configuration of the drill according to the embodiment of the present invention;

FIG. 13 is an exploded perspective view showing a detailed configuration of the drill shown in FIG. 12;

FIG. 14 is a perspective view showing the drill shown in FIG. 12, viewed from another angle;

FIG. 15 is a partial cutaway side view showing another structure of a head portion applicable to the drill according to the embodiment of the present invention;

FIG. 16 is a partial cutaway side view showing a further structure of the head portion applicable to the drill according to the embodiment of the present invention;

FIG. 17 is a perspective view showing an endoscope shown in FIG. 1;

FIG. 18 is a cross-sectional view taken along line A-A of FIG. 12;

FIG. 19 is a sectional view showing a guide tube shown in FIG. 12; and

FIG. 20 is a block diagram showing the method of unilateral biportal endoscopy according to an embodiment of the present invention.

### DETAILED DESCRIPTION OF THE INVENTION

Hereinbelow, an exemplary embodiment of the present invention will be described in detail with reference to the accompanying drawings. Throughout the drawings, the same reference numerals will refer to the same or like parts.

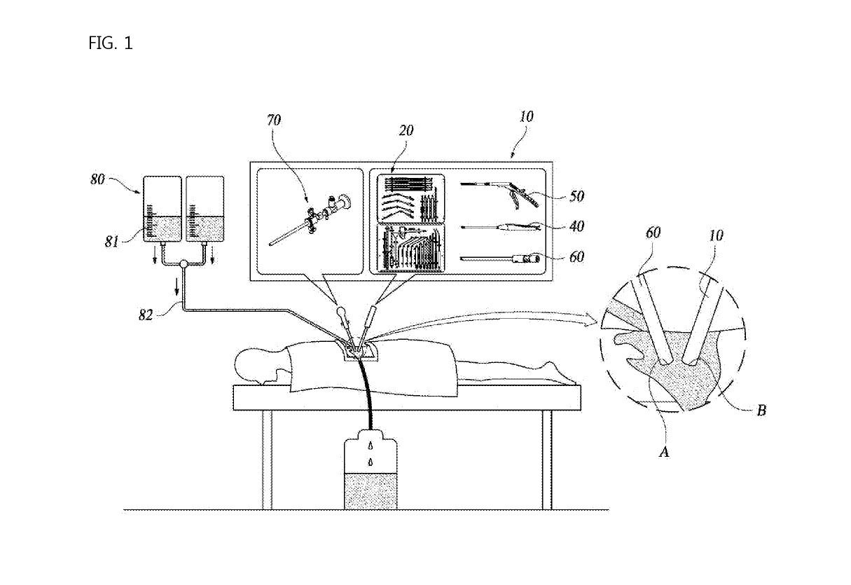

FIG. 1 is a view showing a basic concept of a method of unilateral biportal endoscopy.

The method of unilateral biportal endoscopy is a method whereby two pathways, that is, an endoscopic portal A and a working portal B perforate a surgical site, a surgical instrument set 10 is inserted through the working portal B while an endoscope 70 is inserted through the endoscopic portal A, thereby treating the surgical site. In some cases, the surgical instrument may be inserted through the endoscopic portal A while the endoscope 70 may be inserted through the working portal B.

In particular, a saline solution 81 is injected through the endoscope 70 such that the saline solution is guided to flow through the surgical site, thereby allowing the saline solution to remove residues from the surgical site. The used saline solution is discharged from the body through of the working portal B. As will be described later, the endoscope 70 according to the present embodiment functions to visualize an internal surgical site, as well as to guide the saline solution into the body.

The unilateral biportal endoscopy is characterized in that the surgical instrument and the endoscope approach the surgical site through different pathways, so that a clear field of vision is obtained compared to a conventional method of forming a single incision. Having a clear field of vision is very important factor in spinal surgery.

In addition, since the surgical instrument does not share a pathway with the endoscope 70, a motion of the surgical instrument is relatively free within the pathway, thereby enabling a more efficient surgery.

The surgical instrument set 10 has a very wide range and includes a tool kit 20 including various types of small tools, a radiofrequency probe 40, a K-punch 50, a diamond shaver 60 and the endoscopes 70. The components of the surgical instrument set 10 are selectively used in accordance with the progress of unilateral biportal endoscopic surgery, and all are ergonomically designed.

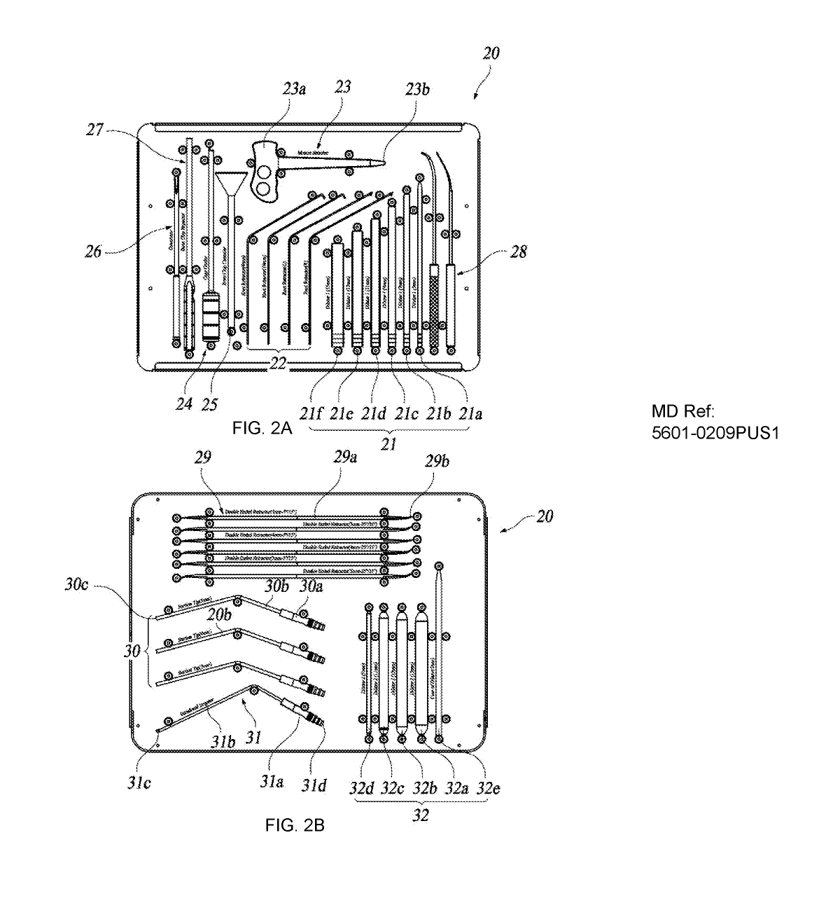

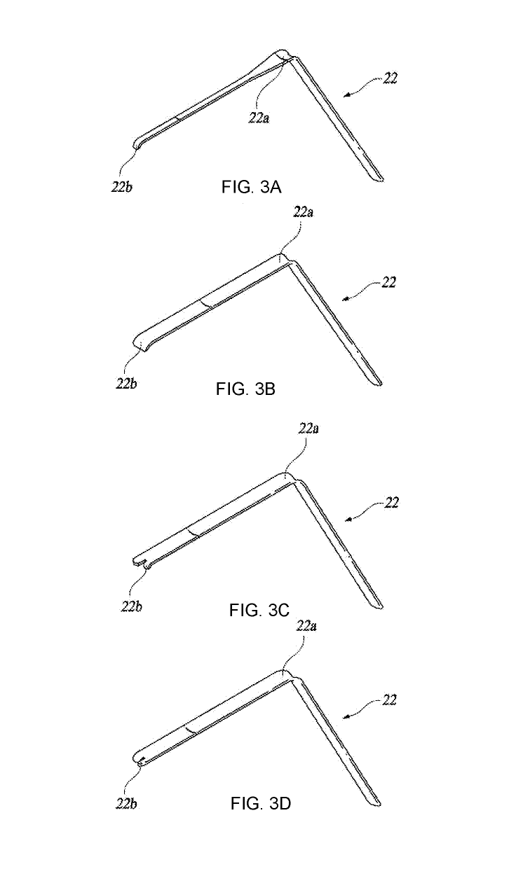

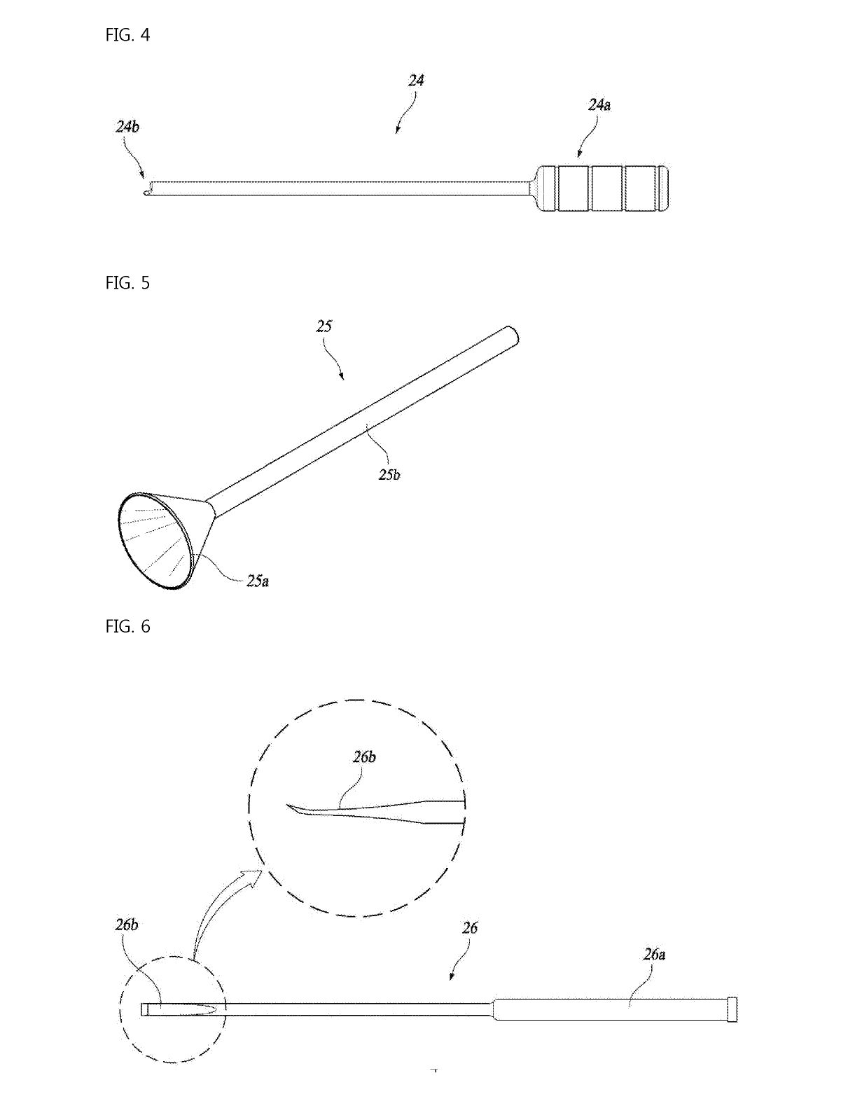

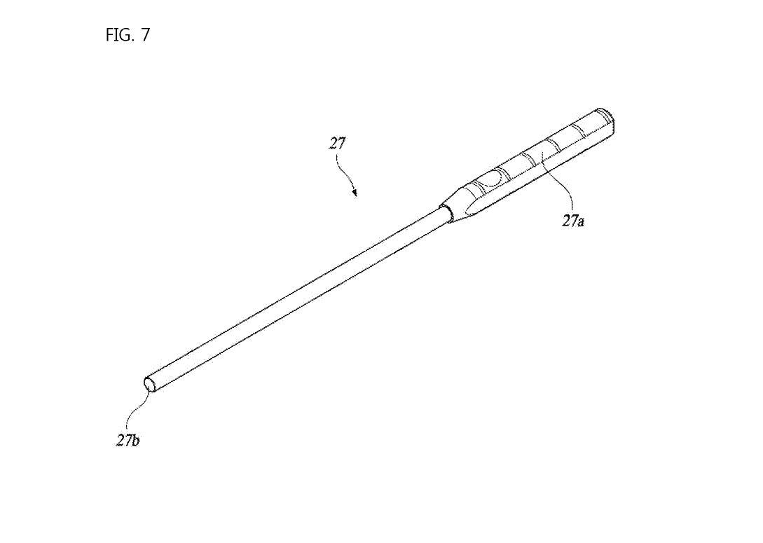

FIGS. 2a and 2b are views showing the tool kit shown in FIG. 1, and FIGS. 3a to 3d are perspective views showing a root retractor 22 shown in FIG. 2a. Further, FIGS. 4 to 8 are views showing the surgical instruments included in the tool kit 20.

As shown in the drawings, the tool kit 20 includes an enlarging tube 21 for enlarging the size of the working portal B and a dilator 32 for retaining the enlarged working portal B.

The enlarging tube 21 is an instrument for enlarging the working portal B by being sequentially inserted thereinto by size in order to secure space for allowing entry of other surgical instruments to the working portal B formed at a surgical site during the unilateral biportal endoscopic surgery. In other words, after making a minimal incision in the skin with a scalpel, the enlarging tubes are stepwisely inserted into the incision to enlarge the same.

The enlarging tube 21 has a hollow tube shape having different diameters and lengths. In the present embodiment, the enlarging tube 21 is provided as six types ranging from a first enlarging tube 21a to a sixth enlarging tube 21f having different sizes. The first to sixth enlarging tubes 21a to 21f are selectively used as required.

The enlarging tube 21 may be provided on the outer circumferential surface thereof with a scale (not shown) marked to indicate the depth of insertion. The first enlarging tube 21a has a sharp front end and serves to enlarge the working portal B and the endoscopic portal A immediately after incision with a scalpel.

The dilator 32 is a bar instrument inserted into the working portal B to retain the working portal B secured by the enlarging tube 21. The dilator 32 includes a first dilator 32a, a second dilator 32b, a third dilator 32c, and a fourth dilator 32d having different sizes as shown in the drawing.

In addition, the tool kit 20 further includes a muscle detacher 23, a double ended retractor 29, a root retractor 22, a suction tip 30, an intradiscal irrigator 31, a cage guider 24, a bone chip cannula 25, an osteotome 26, a bone chip impactor 27, and an end plate remover 28.

The muscle detacher 23 is an instrument for securing an access pathway for an instrument used in the subsequent operation and a working space by detaching muscles from bones at a surgical site in a state of being inserted into the secured incision. In other words, the muscle detacher 23 is inserted between the muscle fibers of the fine muscle rather than cutting the muscle. The muscle detacher 23 has a blade portion 23b and a handle portion 23a. The blade portion 23b has a soft round shape to minimize the skin wound at the surgical site.

The double ended retractor 29 is an instrument being inserted in the working space created by the muscle detacher 23 to detach the nerve root from the bone or the ligamentum flavum or to detach the muscle or ligament.

The double ended retractor 29 is configured such that the angle of tip portions 29b provided at opposite ends thereof are variable, thereby being used for detaching and removing risk factors near the nerves or applying bone wax to a bleeding point during bone bleeding. The tip portion 29b is configured such that an angle thereof is in a range of 5 to 25 degrees, and a width varies to 5.5 mm/4 mm/3 mm. The angle and width of the tip 29b may vary.

The double ended retractor 29 is provided at a center thereof with a handle portion 29a. The handle portion 29a may be provided with a recessed groove for preventing the operator's fingers from slipping or an uneven portion having a predetermined pattern for increasing friction.

The root retractor 22 is an instrument for securing a working space and a constant water pressure in the working space by retracting the muscle and providing a pathway that guides the surgical instruments to be inserted and removed therethrough. As shown in FIGS. 3a to 3d, the root retractor 22 is provided at a center thereof with a first curved portion 22a, and at an end thereof with a second curved portion 22b.

The first curved portion 22a has a curve angle of about 120 degrees, which is an ergonomically and mechanically ideal angle formed between the surgical instrument inserted and a lesion. In addition, the outer edge of the first curved portion 22a has a semi-tubular shape, and the second curved portion 22b has a shape curved in the same direction as the first curved portion 22a or has a half-curved shape to hold the muscle to the nerve root.

The opposite side of the semi-tubular shaped outer edge of the root retractor 22 may serve as the pathway for insertion or removal of the surgical instruments, which detaches soft tissue such as ligaments, etc., or resects or inserts a disc.

The root retractor 22 may have a width of 4 mm/10 mm and the root retractor having a suitable size suitable according to a surgical site may be selectively used. The root retractor 22 helps to open and close the working portal B and maintains the working space and water pressure such that an operator can see clear images of the surgical site. In addition, the root retractor 22 serves to control compression and decompression of the nerve root to enable efficient surgery without damaging the nerve root.

The suction tip 30 is an instrument for sucking a saline solution injected for surgery or the soft tissue as well as tissue debris generated during surgery. During unilateral biportal endoscopic surgery, a constant pressure is required within the body, and thus a constant pressure (e.g., 30 to 50 mmHg) is maintained using the suction tip 30. The suction tip 30 can prevent poor visibility of the surgical field from being caused due to the bone, the tissue debris, etc. during surgery. The suction tip 30 includes a handle portion 30a to which an outlet is connected and a curve-shaped suction pipe portion 30b having at a front end thereof a suction hole 30c. The suction pipe portion 30b may be configured such that a curve angle thereof is about 130 to 150 degrees, and a diameter thereof is 3 to 5 mm.

The suction tip 30 may be used for removing residue, etc. after a space for inserting artificial disc into a disc space is created, or may be used for checking a bleeding site by suctioning a bleeding portion in the peripheral corner of the disc in addition to the disc space.

The suction tip 30 can allow the surrounding debris to be discharged before and after insertion of the artificial disc without remaining within the body, and allow the washing area to be accurately ascertained while providing a sufficient field of vision, thereby enabling quick washing and washing water saving.

The intradiscal irrigator 31 includes a handle portion 31a having a wash water inlet 31d, and a water tube portion 31b curved at a predetermined angle to secure a field of vision of an operator and having at a front end thereof a discharge hole 31c. The water tube portion 31b has a curve angle of about 111 to 130 degrees. When the curve angle is less than 111 degrees, the operator's field of vision is obstructed. Additionally, when the curve angle is greater than 130 degrees, the operator's gaze must be lowered to see the discharge hole 31c.

The cage guider 24 is an instrument for seating a cage (not shown) in the disc space. The cage guider 24 is provided at a first end thereof with a carrying portion 24b on which the cage is placed, and at a second end thereof with a handle portion 24a.

The bone chip cannula 25 is an instrument for colleting the bone chips and inserting them into the cage. The bone chip cannula 25 includes a funnel-shaped colleting portion 25a for concentrating and colleting the bone chips supplied from the outside, and a guide tube portion 25b connected to the colleting portion 25a and extending in the lengthwise direction thereof, the guide tube portion 25b guiding the bone chips to the disc in a state of reaching a surgical site.

The osteotome 26 is an instrument for cutting unnecessary bones during surgery. The osteotome 26 is provided at a front end thereof with a cutting blade portion 26b cutting the bone, and at an opposite end to the tip end thereof with a handle portion 26a.

The bone chip impactor 27 is an instrument for impacting on the artificial disc inserted into the disc space or the collected bone material so as to be seated in a precise position. The bone chip impactor 27 is provided at a front end thereof with a tip portion 27b being in contact with a target to be impacted thereon, and at an opposite end to the tip end thereof with a handle portion 27a.

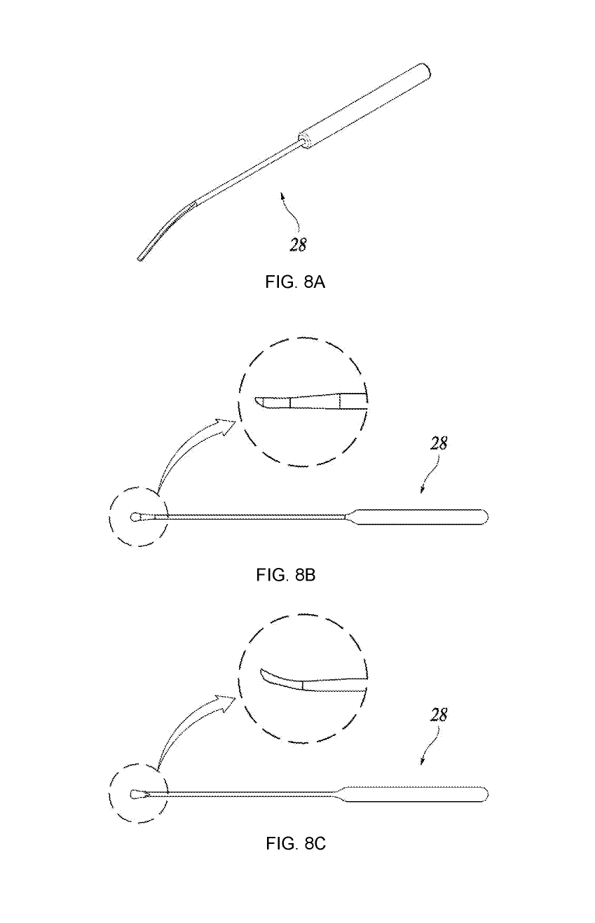

Further, the end plate remover 28 is an instrument for removing the end plate located between the vertebrae and the disc, and is curved at a front end thereof in a hook shape. Since the tip end of the curved end plate remover 28 has a hook shape, each approach to and removal of the end plate located between the vertebrae and the disc is possible. As shown in FIGS. 8a, 8b, and 8c, the tip end of the end plate remover 28 may vary in shape.

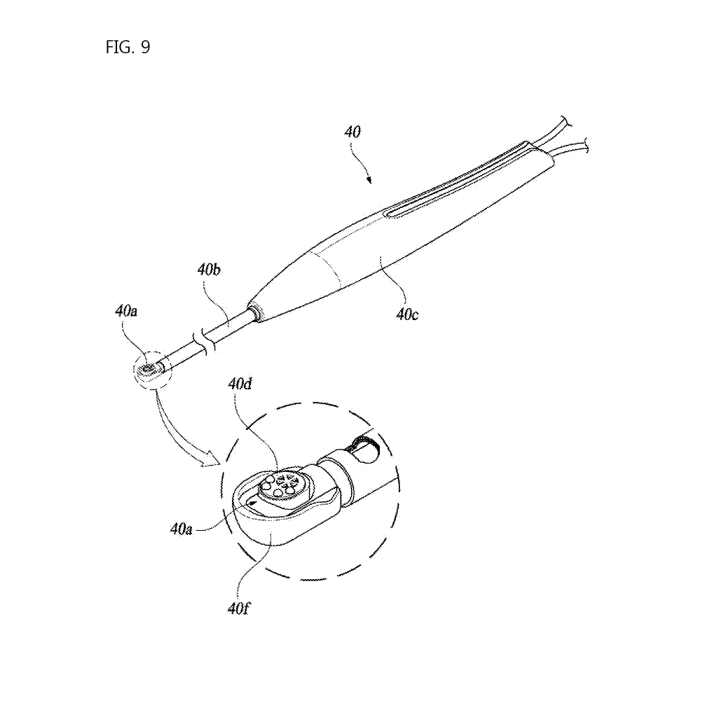

FIG. 9 is a partial perspective view showing the radio frequency probe 40 shown in FIG. 1.

The radiofrequency probe 40 is an instrument for heating and removing the soft tissue, disc, epidural fat, and ligaments. While a conventional radiofrequency probe is problematic in that a tip thereof where radiofrequency is generated is in direct contact with a surgical site and thus the surrounding nerve is damaged, the radiofrequency probe 40 according to the present invention has a safety protrusion (not shown) whereby no damage to normal tissue is caused.

The radiofrequency probe 40 includes an insertion rod 40b inserted into the body so as to reach a surgical site, an electrode tip 40a provided at a front end of the insertion rod 40b and outputting radiofrequency heat by being applied with electric power from outside, and the safety protrusion formed on the surface of the electrode tip 40a and separating the surface of the electrode tip 40a from the body tissue to prevent thermal damage.

In addition, the radiofrequency probe 40 may further include an electric power wire supplying electric power to the radiofrequency probe 40, and a discharge tube extending from the outside of a casing 40c and discharging a saline solution in the body therefrom.

Moreover, the electrode tip 40a may be detachably fitted into the insertion rod 40b and includes a shield portion 40f. The shield portion 40f is a soft round-shaped member for minimizing damage to the body tissue and facilitating insertion when the electrode tip 40a is inserted into the body. The shield portion 40f also serves to block heat of plasma from being transferred to normal tissue.

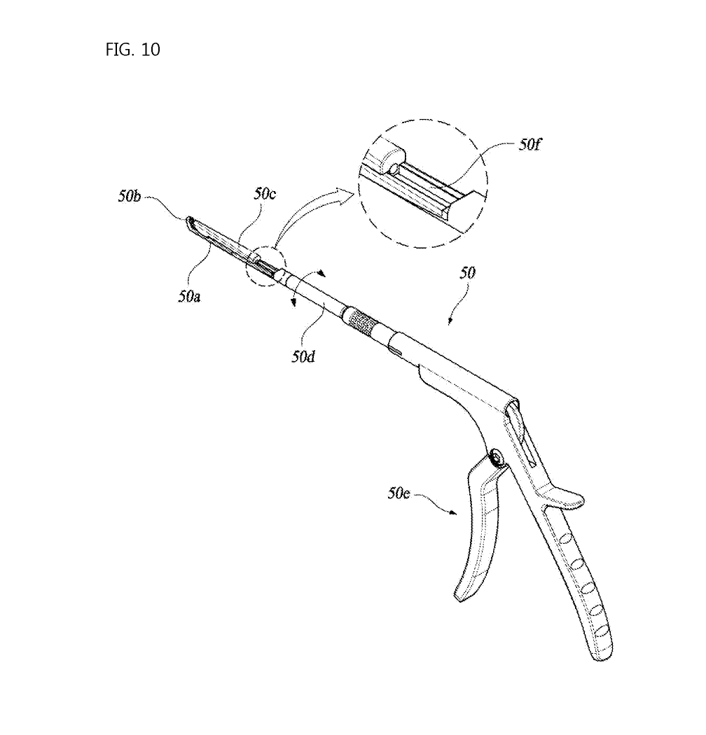

FIG. 10 is a perspective view showing the K-punch 50 shown in FIG. 1.

The K-punch 50 is an instrument for detaching and removing the bone, ligamentum flavum, soft tissue, etc. and includes an entry rod 50a, a slider 50c, a rotary shaft 50d, a pushing rod 50f, and a handle portion 50e.

The entry rod 50a is a member being inserted into the body so as to reach a surgical site at a front end thereof, and is provided at the front end thereof with a retaining step portion 5b. Further, the slider 50c is slidably engaged with a side of the entry rod 50a and moves forward and backward with respect to the retaining step portion 50b. The slider 50c is pressed and moved to the retaining step portion 50b in a state in which a target to be removed is positioned between the retaining step portion 50b and the slider 50c, whereby the target to be removed is physically fixed.

The rotary shaft 50d is fixed to the rear side of the entry rod 50a, and is rotated by an operator's operation as required during surgery such that the direction of the retaining step portion 50b is controlled. As such, by provision of the rotary shaft 50d, the handle portion 50e is operable at a comfortable angle regardless of the position of tissue to be removed.

The pushing rod 50f is fixed at a front end thereof to the slider 50c and extends from a rear end thereof to the rear side of the rotary shaft 50d, the pushing rod being configured to move forward to press and move the slider 50c to the retaining step portion 50b when the handle portion 50e is manipulated.

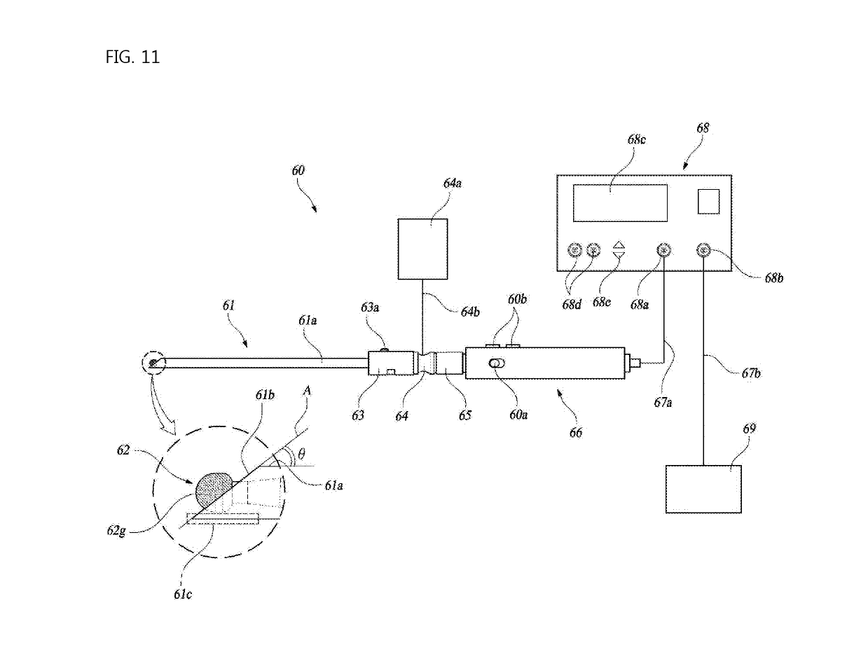

FIG. 11 is a view showing an overall configuration of the diamond shaver 60 applicable to the method of unilateral biportal endoscopy according to the embodiment of the present invention.

As shown in the drawing, the diamond shaver 60 includes a drill 61 extending in the lengthwise direction thereof and partially inserted into the body during surgery, a handpiece 66 connected to a rear end of the drill 61, a controller 68 outputting a control signal of the handpiece 66, and a foot switch 69 connected to the controller 68 and outputting a switching signal when the foot switch 69 is operated by the operator's foot.

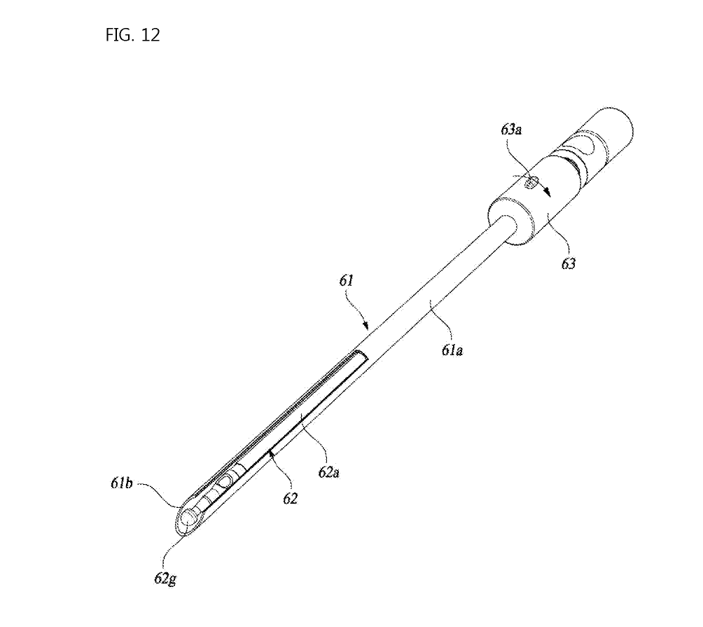

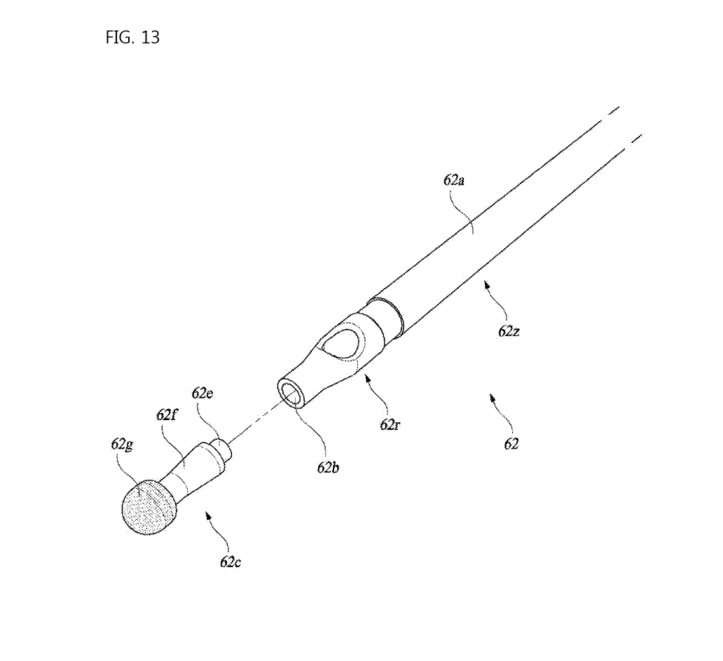

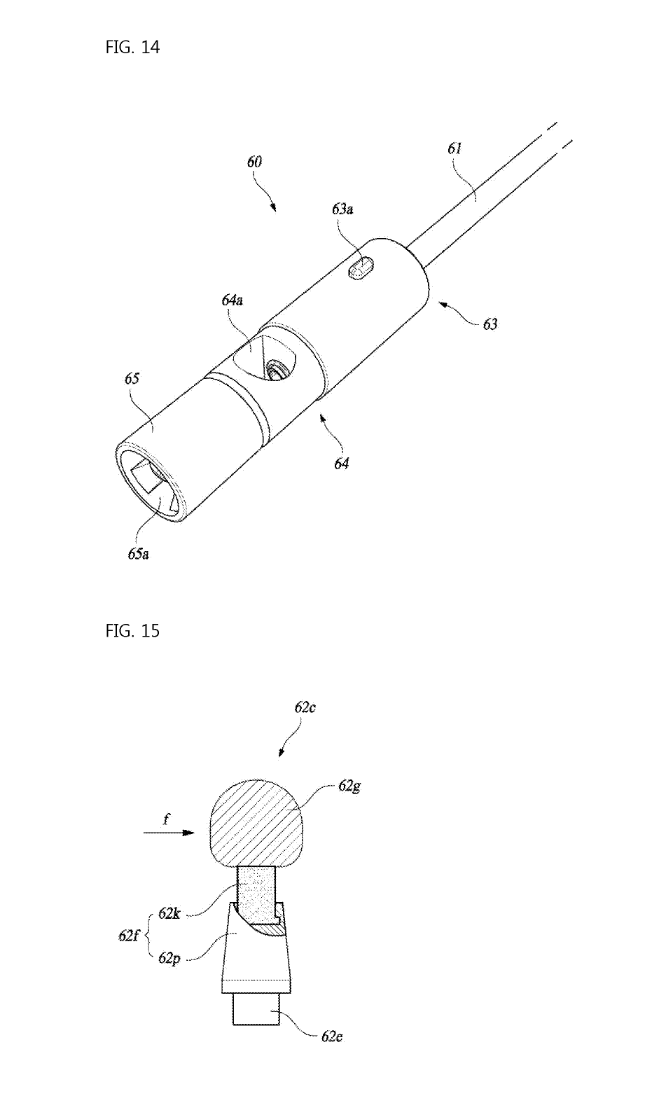

The drill 61 includes an outer tube 61a, a drill body 62 provided inside the outer tube 61a, a tube holder 63, a fluid guiding portion 64, and a handpiece adapter 65, wherein the tube holder, the fluid guiding portion, and the handpiece adapter are sequentially provided at a rear end of the outer tube 61a. The drill 61 may have structures shown in FIGS. 12 to 14.

The drill 61 will be described in detail below with reference to FIGS. 12 to 14.

First, the outer tube 61a is a hollow tube-shaped member having a predetermined inner diameter and extending in the lengthwise direction thereof, and has at a front end thereof an inclined opening 61b. The inclined opening 61b is a portion formed by cutting the front end of the outer tube 61a so as to have an inclination angle θ Shown in FIG. 11. The inclination angle θ between a plane A including the inclined opening 61b and the lengthwise axis of the outer tube 61a is 35 degrees to 45 degrees, preferably 38 degrees.

As such, the inclined opening 61b is applied to the front end of the outer tube 61a whereby a burr 62g that will be described later is partially covered on the outer circumferential surface thereof even when the burr 62g is exposed to the outside of the outer tube 61a.

The portion that partially covers the burr 62g is a blocking portion 61c serving to protect a portion that should not be cut during surgery. For example, when the bone adjacent to nerve tissue is cut and removed, the nerve tissue is covered by the blocking portion 61c such that the burr 62g is blocked from touching the nerve tissue. In other words, the nerve tissue is separated from the burr 62g. In general, since the nerves and blood vessels are intricately entangled with the vertebrae or surround the same, absence of blocking portion 61c may cause nerve damage or rupture of the blood vessels during surgery.

The drill body 62 provided inside the outer tube 61a is rotated by receiving a rotational force transmitted from the handpiece 66 and shaves a target. The drill body 62 includes a rotatable shaft portion 62z and a head portion 62c, and is distanced from the inner circumferential surface of the outer tube 61a.

The rotatable shaft portion 62z has a hollow shaft 62a extending in the outer tube 61a in the lengthwise direction thereof and a tip portion 62r. The hollow shaft 62a is connected to the handpiece 66 and is rotated clockwise or counterclockwise by receiving a rotational force transmitted from the handpiece 66. A rotational direction of the hollow shaft 62a may be manipulated by the controller 68, the handpiece 66, or the foot switch 69.

The tip portion 62r is integrally formed with a front end of the hollow shaft 62a and is provided with a coupling hole 62b such that the tip portion 62r and the head portion 62c are coupled with each other. In other words, the tip portion 62r serves as an adapter for connecting the hollow shaft 62a and the head portion 62c to each other.

The head portion 62c includes a connection portion 62e mounted into the coupling hole 62b of the tip portion 62r, a burr supporting portion 62f integrally formed with the connection portion 62e, and a burr 62g coupled with the burr supporting portion and grinding bones, hard tissue, etc.

The connection portion 62e is fitted into the coupling hole 62b and serves to transmit a rotational force of the hollow shaft 62a to the burr supporting portion 62f. The type of the tip portion 62r, and the configuration and engagement manner of the connection portion 62e may vary as long as such a role is achieved. In addition, the head portion 62c may be replaced with respect to the tip portion 62r as needed. The burr 62g is a member on which fine diamond powder (not shown) is distributed, and serves to grind a target to be removed, such as bone spurs, hard tissue, etc. in a state of being in contact therewith. The diamond powder is a cutting diamond having a grain size of approximately 30 to 200 mesh. In addition, the shape of the burr 62g may vary, and may be a spherical shape, a cylindrical shape, a disc shape, a triangular pyramid shape, etc.

Meanwhile, the tube holder 63 fixed to the rear side of the outer tube 61a is formed by molding a synthetic resin, and has on the outer circumferential surface thereof a protrusion 63a. The protrusion 63a serves to indicate the opening direction of the inclined opening 61b and to prevent the tube holder 63 from slipping when the tube holder 63 is rotated and manipulated.

Due to a position of the protrusion 63a, an operator of the diamond shaver 60 can ascertain the direction of the inclined opening 61b being invisibly inserted into the body, that is, a position of the blocking portion 61c. Although an operator generally monitors a surgical point through various imaging systems during surgery, due to the protrusion 63a, the operator can easily ascertain which direction the burr 62g is exposed without turning his or her head to a monitor.

Further, the rear end of the hollow shaft 62a further extends rearward through the center axis of the tube holder 63 so as to receive a rotational force from the handpiece 66. The length of the hollow shaft 62a in the rearward direction and the engagement manner thereof with the handpiece 66 may vary according to the type of handpiece 66 used.

The fluid guiding portion 64 provided at a rear side of the tube holder 63 serves as a passage for discharging outside a saline solution, body fluid, or blood which is stagnated at a surgical site and its peripheries during surgery. For this purpose, an opening 64a is provided at a side of the fluid guiding portion 64. The opening 64a is a hole to which a connection tube (64b in FIG. 11) is connected. The connection tube 64b is fixed at an end thereof to the opening 64a using a separate adapter (not shown).

The handpiece adapter 65 holds and fixes the handpiece 66, and has a fastening hole 65a being open rearward. The front end of the handpiece 66 is fitted into the fastener 65a so that the handpiece adapter 65 and the handpiece 66 are coupled with each other. The shape and the fastening manner of the handpiece adapter 65 may vary according to the structure of the handpiece 66.

For example, magnets may be arranged inside the handpiece adapter 65 such that the handpiece 66 is magnetically coupled to the handpiece adapter 65, or the handpiece adapter 65 and the handpiece 66 may be provided with a groove and a protrusion that correspond to each other such that the handpiece adapter 65 and the handpiece 66 are coupled with each other in a fitted manner.

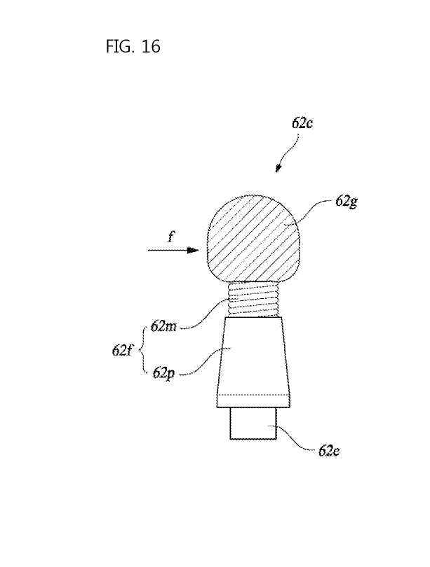

Meanwhile, the structure of the head portion 62c may vary, and may have a structure shown in, for example, FIG. 15 or 16.

FIGS. 15 and 16 are partial cutaway side views showing further structures of a head portion applicable to the drill 61 according to the embodiment of the present invention.

The head portion 62c shown in FIG. 15 is configured such that the burr supporting portion 62f is composed of a tapered body 62p and a torsion rubber 62k. The tapered body 62p is a member fixed to the connection portion 62e, and is coupled with a lower end of the torsion rubber 62k in the drawing.

The torsion rubber 62k is an elastic member made of rubber and having a predetermined diameter, and is coupled at an upper end thereof with the burr 62g. The coupling manner of the torsion rubber 62k and the burr 62g may be variously implemented. The torsion rubber 62k transfers a rotational force transferred from the connection portion 62e to the burr 62g.

Particularly, since the torsion rubber 62k has an elastic force, when a force is applied to the torsion rubber 63k in the direction of an arrow f, the torsion rubber 63k is elastically deformed to some extent (within a range not touching the blocking portion 61c), thereby buffering impact upon cutting.

A coil spring 62m shown in FIG. 16 has the same purpose as the torsion rubber 62k. In other words, the coil spring 62m serves to transfer a rotational force transferred from the handpiece 66 to the burr 62g, and is elastically deformed in response to a lateral force laterally applied in the direction of the arrow f, thereby buffering impact upon cutting.

As shown in FIG. 11, the drill 61 having the above configuration is used for surgery in a state of being connected with the handpiece 66, the controller 68, and the foot switch 69.

Referring to FIG. 11, the controller 68 is provided with a plurality of control terminals 68a and 68b, a direction control switch 68d, a speed control switch 68e, a display portion 68c, and an on/off switch 68.

The control terminals 68a and 68b are connected with the handpiece 66 and the foot switch 69 through signal cables 67a and 67b, and receive and transmit a signal. The signal is a control signal related to a rotational speed or a rotational direction of the burr 62g.

The direction control switch 68d is a switch for determining the rotational direction of the burr 62g. The rotational direction of the burr 62g is switched clockwise or counterclockwise by the direction control switch 68d. Further, the speed control switch 68e is a switch for controlling the rotational speed of the burr 62g. The rotational speed of the burr 62g is optimally controlled by the speed control switch 68e.

The control signal is outputted to the control terminal 68a through the direction control switch 68d or the speed control switch 68e and is then transmitted to the handpiece 66 through the signal cable 67a. The on/off switch 68f is a switch for turning on or off the controller 68.

The display portion 68c serves to display the rotational speed and the rotational direction of the burr 62g currently operating in real time, or serves to indicate a set rotational speed of the burr 62g.

The foot switch 69 is a switch for selecting rotation and stop of the burr 62g, for example, in a state in which the rotational speed or the rotational direction of the burr 62g is set. Since it is inconvenient to manipulate rotation of the burr 62g in a state in which an operator holds the surgical instruments with both hands, the foot switch 69 enhances operator comfort during surgery.

Meanwhile, the handpiece 66 may be provided with various manipulation switches 60b and an on/off switch 60a. The on/off switch 60a is a switch for turning on and off a motor (not shown) mounted in the handpiece 66. Further, the manipulation switch 60b is a switch for controlling the rotational direction and the rotational speed of the burr 62g (It is to be noted that mounting the motor in the handpiece is known in the art).

In terms of driving the burr 62g, the controller 68, the handpiece 66, and the foot switch 69 form a parallel arrangement.

The operation of the diamond shaver 60 having the above configuration is as follows. First, a front end of the drill 61 is advanced toward a target point where a target site to be removed in the body is located. When the burr 62g of the drill 61 reaches a target point, the controller 68 is controlled to rotate the burr 62g with the burr 62g facing a target part to be cut. Herein, the rotational direction and rotational speed of the burr 62g are appropriately determined in accordance with necessity.

During surgery, an operator may control operation of the burr 62g by using the foot switch 69. When a cutting process as described above is completed, the drill 61 is taken out of the body and then surgery is completed.

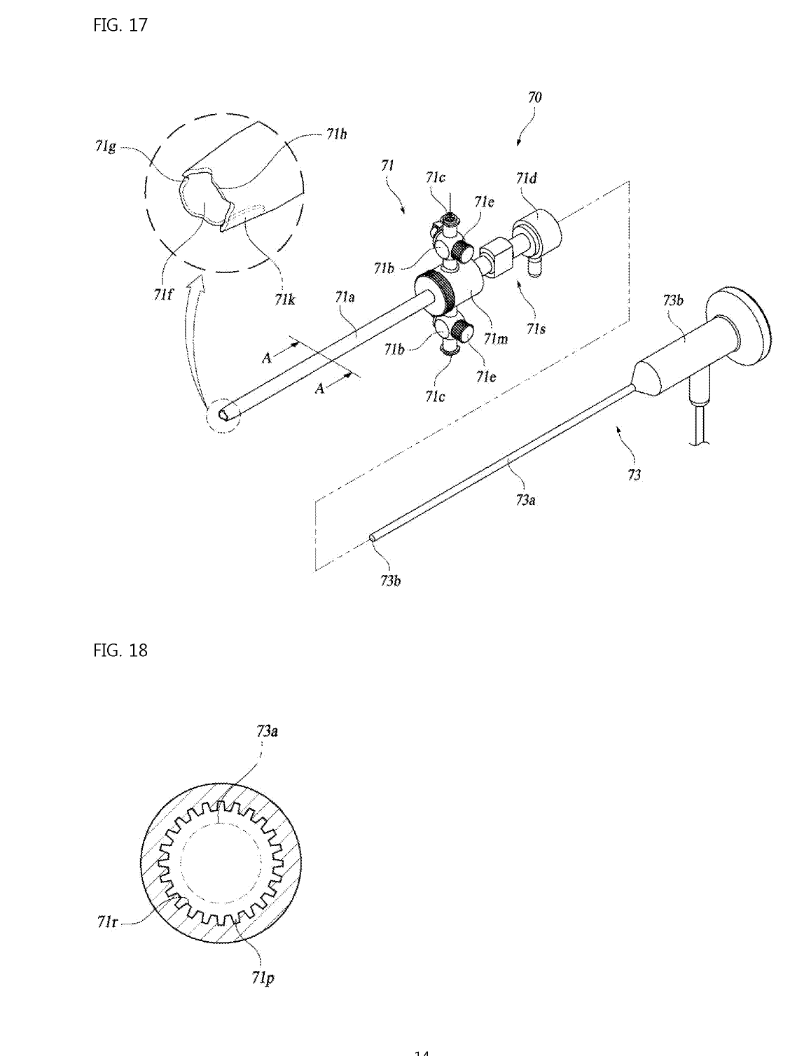

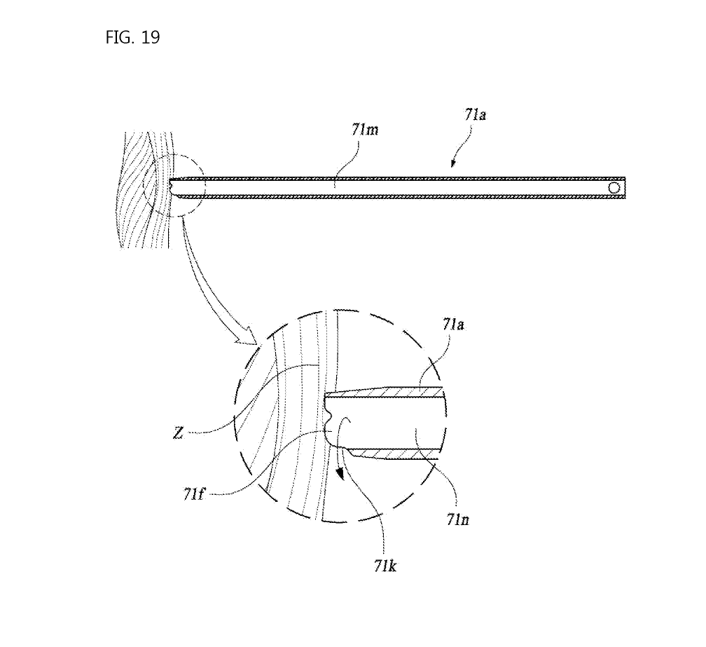

FIG. 17 is a perspective view showing the endoscope 70 shown in FIG. 1, and FIG. 18 is a cross-sectional view taken along line A-A of FIG. 17. Further, FIG. 19 is a sectional view showing the guide tube 71a shown in FIG. 17. The endoscope 70 includes a sheath mechanism 71 and an endoscope camera 73.

The endoscope camera 73 is a device for identifying and capturing an image of a surgical site in the body, and includes a flexible probe 73a extending in the lengthwise direction thereof and having an optical fiber cable therein, A lens 73b provided at a front end of the probe 73a, and a lens barrel 73c provided at a rear end of the lens 73b.

The endoscope camera 73 may further include an imaging control device for capturing and recording images, a light source connected to a guide cable for illuminating a imaging site, the guide cable for transporting light to a distal end of the endoscope 70 for emitting light to the imaging site, and an endoscope tray storing the endoscope camera 73 and facilitating movement of the endoscope camera 73.

The sheath mechanism 71 is combined with the endoscope camera 73 to constitute a single endoscope 70 and serves to support the endoscope camera 73 during surgery whiling secure a field of vision. The reason why the sheath mechanism 71 is used is that the probe 73a of the endoscope camera is very thin and tends to be curved, and thus the lens 73b may not be allowed to reach a target point in the body. Another important function of the sheath mechanism 71 is to guide a saline solution to a target point.

The sheath mechanism 71 includes a guide tube 71a, a damping chamber 71m, a valve body 71b, and an adapter portion 71s.

The guide tube 71a is a hollow tube-shaped member that extends in the lengthwise direction thereof, and a first end thereof reaches a surgical site in the body when in use. The material of the guide tube 71a may vary and may be made of, for example, stainless steel or a synthetic resin including polypropylene.

The length of the guide tube 71a may vary as required. The guide tube 71a is inserted into the body through the portal secured by the enlarging tube 21.

In particular, the guide tube 71a is provided on an inner circumferential surface thereof with a plurality of guide grooves 71p. The guide grooves 71p extend in the lengthwise direction of the guide tube 71a and serve to guide a saline solution supplied from the outside to an outlet 71f.

As shown in FIG. 18, linear protrusions 71r are provided between the guide grooves 71p, respectively. The linear protrusions 71r are arranged in parallel with the guide grooves 71p, and a plurality of the protrusions are arranged in parallel to form the guide grooves 71p. The linear protrusions 71r and the guide grooves 71p are arranged in the circumferential direction of the guide tube 71a to be distanced from each other at predetermined intervals.

Additionally, the linear protrusions 71r are in partial contact with an outer circumferential surface of the probe 73a inserted into a space portion 71n of the guide tube 71a and to thereby support the probe 73a. The diameter of a virtual cylinder connecting the upper ends of the linear protrusions 71r is greater than the diameter of the probe 73a. Thus, the probe 73a can move vertically and horizontally in the space portion 71n and freely slide in the lengthwise direction thereof.

Furthermore, the guide tube 71a is provided at a front end thereof with a plurality of projecting portions 71h and a plurality of depressed portions 71g. The projecting portions 71h projects in a direction of the front end of the guide tube 71a, that is, in a direction in which a saline solution is discharged, and the depressed portions 71g are depressed in a direction opposite thereto. In particular, the projecting portions 71h and the depressed portions 71a are repeatedly provided in a wave pattern in the circumferential direction of the guide tube 71a.

The projecting portions 71h and the depressed portions 71a serve to guide a saline solution discharged from the guide tube 71a to flow out in the radial direction of the guide tube 71a. For example, when the front end of the guide tube 71a is clogged with the muscle, the saline solution is allowed to be supplied through the depressed portions 71a, or is imparted with directionality for securing a field of vision.

In addition, the guide tube 71a is provided with a side slit 71k formed on the side of the front end of the guide tube 71a. The side slit 71k serves to control the flow direction of a saline solution. In other words, during unilateral biportal endoscopic surgery, the flow direction of the saline solution is controlled, whereby the lens 73b is easily cleaned while the saline solution flows by gravity, thereby securing a field of vision of the endoscope 70.

The side slit 71k serve as a passage for a saline solution. For example, as mentioned above, the side slit 71k is provided to prevent a case where the depressed portions 71a of the guide tube 71a are clogged with tissue such as muscle Z and thus the saline solution is not efficiently discharged, and is provided to impart directionality to the saline solution to secure a field of vision.

The saline solution introduced into the guide tube 71a is discharged through the side slit 71k by gravity and washes away tissue or blood of the affected area, thereby securing a field of vision.

The adapter portion 71s serves to maintain a position of the endoscope camera 73 with respect to the sheath mechanism 71, and has a holder 71d for supporting the endoscope camera 73. The guide tube 71a is open at a rear end thereof to the rear side of the holder 71d. When the probe 73a is fully inserted into the guide tube 71a through the holder 71d, the endoscope camera 73 is supported by the holder 71d and thus is prevented from being separated backward.

Meanwhile, the damping chamber 71m is a space communicating with the rear end of the guide tube 71a, and serves to receive a saline solution supplied through an inlet 71c and a valve body 71b, store the same therein, and transfer the stored saline solution to the guide tube 71a.

By provision of the damping chamber 71m, deviation in the flow rate of a saline solution supplied to the guide tube 71a is kept as low as possible. When the damping chamber 71m is absent, a change in the flow rate of the saline solution supplied through a saline solution supply tube (reference numeral 82 in FIG. 1) is immediately reflected in the guide tube 71a. The capacity of the damping chamber 71m may vary as required.

Two valve bodies 71b are provided at the periphery of the damping chamber 71m, and each of the valve bodies 71 is provided with a flow control valve 71e. The flow control valve 71e serves to control the flow rate of a saline solution passing through the valve body 71b and is manipulated by an operator.

Reference numeral 71c denotes an inlet to which the saline solution supply tube 82 is connected. The saline solution having flowed through the saline solution supply tube 82 reaches the affected area through the inlet 71c via the valve body 71b, the damping chamber 71m, and the guide tube 71a.

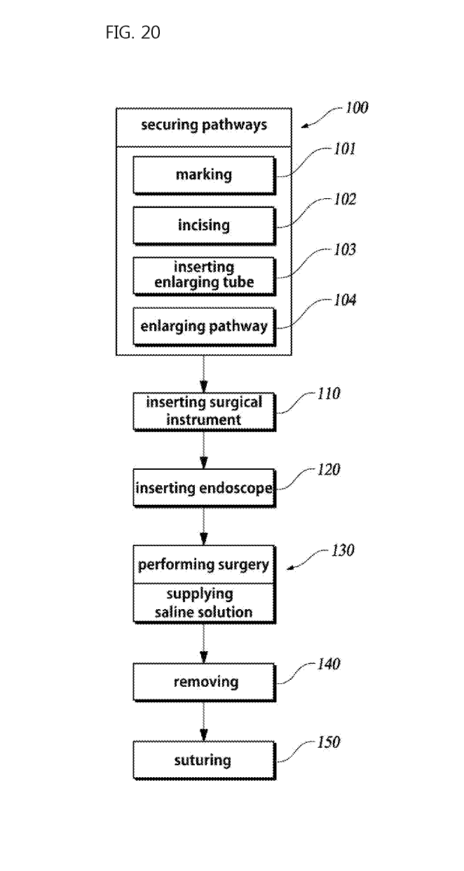

FIG. 20 is a block diagram showing the method of unilateral biportal endoscopy according to the embodiment of the present invention.

As shown in the drawing, the method of unilateral biportal endoscopy according to the present embodiment includes a step of securing pathways S100, a step of inserting a surgical instrument S110, a step of inserting an endoscope S120, a step of performing surgery S130, a step of removing S140, and a step of suturing S150.

The step of securing the pathways S100 is a process of forming two pathways extending toward a surgical site in the patient's body, that is, the working portal B and the endoscopic portal A, and includes marking S101, incising S102, enlarging tube inserting S103, and pathway enlarging S104.

First, the marking S101 is a process of marking points at which the working portal B and the endoscopic portal A are formed on the skin on the vertebral region of a patient lying in a prone position. In other words, entrances through which an instrument, such as the tool kit 20 or the diamond shaver 60 from the surgical instrument set is inserted are marked. In particular, two marking points must be distanced from each other. The marking points vary depending on the location of a surgical site. When a lesion is located in a deep position, the distance between the two marking points is increased.

The working portal B and the endoscopic portal A are independent pathways to each other, and are configured to meet with each other at a lesion site in the body whereas the entrances thereof are separated from each other, thereby forming substantially the sides of a triangle.

When the marking S101 is completed, the incising S103 is performed. For example, the incising S103 is a process of making incisions on marking portions using a scalpel, whereby the entrance through which the enlarging tube 21 is inserted is opened. Herein, the incision length may be about 5 mm.

Subsequently, the enlarging tube inserting S103 is a process of forming a straight pathway toward a surgical site by inserting the enlarging tube 21 into the body using the incision opened through the incising S102 as an entrance. Of course, the enlarging tube 21 used first is the first enlarging tube 21a having the smallest diameter.

The pathway enlarging S104 is a process of enlarging the diameter of the pathway by using enlarging tubes having different sizes. For example, in a state in which the first enlarging tube 21a is inserted into the body, the second enlarging tube 21b is inserted thereover and then the first enlarging tube 21a is taken out. Thereafter, the third enlarging tube 21c is inserted over the second enlarging tube 21c and then the second enlarging tube 21b is taken out in such a manner that the diameter of the pathway is increased.

The pathway enlarging S104 may be applied to both the endoscopic portal A and the working portal B. Needless to say, the diameter of the working portal B through which the surgical instrument set is inserted should be relatively large.

As described above, the endoscopic portal A and the working portal B formed through the step of securing the pathways S100 are distanced from each other on the patient's epidermis but meet with each other at a surgical site in the body.

Subsequently, the step of inserting the surgical instrument S110 is a process of inserting the surgical instrument required for surgery through the working portal B secured through the step of securing the pathways S100. In other words, it is a process of inserting the required surgical instruments according to the progress of surgery. The radiofrequency probe 40, the K-punch 50, and the diamond shaver 60 as well as the tool kit 20 are selectively inserted through the working portal B as required.

The step of inserting the endoscope S110 is a process of inserting the endoscope 70 through the secured endoscopic portal A. Of course, the sheath mechanism 71 and the lens 73b of the endoscope camera 73, which constitute the endoscope 70, must reach a lesion site.

Then, the step of performing the surgery S130 is performed. The step of performing the surgery S130 is a process of performing surgery using the surgical instrument set 100 inserted into the working portal B while monitoring a surgical site through the endoscope 70.

The step of performing the surgery S130 is a process of actually performing treatment on a surgical site to be treated in the body. As the treatment progresses, the required surgical instruments are inserted into the body through the working portal B. Of course, a surgery status is continuously monitored through the endoscope 70 during surgery.

In particular, during the step of performing the surgery S130, saline solution supplying is performed. The saline solution supplying is a process of supplying a saline solution supplied from the outside to a surgical site and discharging materials to be discharged generated during surgery from the body. As described above, the saline solution is guided through the guide tube 71a of the sheath mechanism 71. The injected saline solution allows debris at a surgical site and tissue removed to be discharged outside, and particularly allows fine powder removed by using the diamond shaver 60 to be efficiently discharged.

Subsequently, the step of removing S140 is a process of removing the used surgical instrument and the endoscope 70 from the body. Herein, the surgical instrument may be removed prior to removing the endoscope 70. For example, the endoscope camera 73 is used to check and identify a surgical site prior to removal thereof.

When the step of removing S140 is completed, the step of suturing S150 of suturing the entrances of the working portal B and the endoscopic portal A is performed, whereby surgery is completed.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A method of unilateral biportal endoscopy, the method comprising:
securing pathways for a working portal and an endoscopic portal that extend toward a surgical site in the body of a patient and are distanced from each other;
inserting a surgical instrument required for surgery into the secured working portal;
inserting an endoscope into the endoscopic portal;
performing surgery using the surgical instrument inserted into the working portal while monitoring the surgical site through the endoscope;
removing the surgical instrument and the endoscope after the performing the surgery; and
suturing entrances of the working portal and the endoscopic portal.

2. The method of claim 1, wherein the securing the pathways includes:
marking positions of the entrances of the working portal and the endoscopic portal on the skin of the patient;
incising marking portions marked by the marking;
inserting an enlarging tube into the body through an incision opened by the incising, thereby forming a pathway extending toward the surgical site; and
enlarging the pathway to enlarge a diameter of the pathway by using enlarging tubes having different sizes.

3. The method of claim 1, further comprising:
supplying a saline solution from outside to the surgical site and discharging materials generated at the surgical site from the body, during the performing the surgery.

4. The method of claim 1, wherein the working portal and the endoscopic portal are configured such that the entrances thereof are distanced from each other, and the portals extend into the body to be close to each other such that ends thereof meet with each other at the surgical site.

5. The method of claim 4, wherein an angle between the working portal and the endoscopic portal is equal to or less than 90 degrees.

6. A diamond shaver used in unilateral biportal endoscopy, the diamond shaver being configured to be inserted into a working portal and to shave and remove a target located at the surgical site, the working portal extending toward a surgical site of a patient together with an endoscopic portal, the diamond shaver comprising:
a drill including an outer tube having an inclined opening inclined at a front end thereof to form an acute angle with respect to a lengthwise direction of the outer tube, a tube holder fixed to a rear end of the outer tube, a handpiece adapter provided at a rear side of the tube holder, and a drill body rotatably provided in the outer tube, the drill body including a rotatable shaft portion extending in a lengthwise direction thereof, and a head portion fixed to a front end of the rotatable shaft portion and having a burr embedded with diamond powder;
a handpiece detachably coupled to the handpiece adapter and manipulated by an operator;
a controller connected to the handpiece and outputting a control signal; and
a foot switch connected to the controller and manipulated by an operator's foot.

7. The diamond shaver of claim 6, wherein the burr is configured to be partially exposed to outside of the outer tube through the inclined opening, such that the burr is brought into contact with a target to be removed.

8. The diamond shaver of claim 6, wherein the rotatable shaft portion is provided with:
a hollow shaft extending in a lengthwise direction thereof; and
a tip portion provided at a front end of the hollow shaft and provided with a coupling hole such that the tip portion is coupled with the head portion, and
the head portion is provided with:
a connection portion detachably coupled with the coupling hole; and
a burr supporting portion integrally provided with the connection portion and supporting the burr.

9. The diamond shaver of claim 6, wherein the burr supporting portion is provided with:
a tapered body receiving a rotational force from the adapter; and
an elastic supporting member being elastically deformable and connecting the tapered body and the burr to each other.

10. The diamond shaver of claim 6, wherein the elastic supporting member includes:
a cylindrical torsion rubber or a coil spring having a predetermined diameter.

11. The diamond shaver of claim 6, wherein a fluid guiding portion is provided between the tube holder and the handpiece adapter, and allows a saline solution to pass therethrough.
